(19) United States
(12) Patent Application Publication
(10) Pub. No.: US 2007/0054872 A1
(43) Pub. Date: Mar. 8, 2007

(54) REGULATABLE OR CONDITIONAL EXPRESSION SYSTEMS

(75) Inventors: Thomas W. Reppen, Madison, WI (US); David L. Lewis, Madison, WI (US)

Correspondence Address: MIRUS CORPORATION, 505 SOUTH ROSA RD, MADISON, WI, 53719, US

(73) Assignee: Mirus Bio Corporation, Madison, WI (US)

(21) Appl. No.: 11/466,797

(22) Filed: Aug. 24, 2006

Related U.S. Application Data
(60) Provisional application No. 60/711,080, filed on Aug. 24, 2005.

(51) Int. Cl. A61K 48/00; C07H 21/02

(52) U.S. Cl. 514/44; 435/455; 536/23.1

(57) ABSTRACT

Endogenous gene regulation mechanisms together with microRNAs expressed in many organisms can be used to provide regulated or conditional expression of transgenes by placing an appropriate sequence, a microRNA binding site, within the transcribed gene. This microRNA-dependent transcription regulation can be further regulated using microRNA inhibitors.

### CROSS-REFERENCE TO RELATED APPLICATIONS

This application claims the benefit of U.S. Provisional Application No. 60/711,080, filed Aug. 24, 2005.

### BACKGROUND OF THE INVENTION

Regulation of transgene expression in mammals would be advantageous in both experimental and gene therapy settings. In experimental settings, the ability to express a transgene at specific times and in specific tissues would enable detailed analyses of the effects of expression in a temporal and spatial context. This is especially important in determining a gene's function under specific conditions. For example, many genes involved in disease processes are misregulated. The interest of the investigator is to determine which of the genes are primarily responsible for the disease phenotype versus those that are secondarily affected. Once the primary genes are identified, a clearer path is revealed for drug development and possible therapeutic intervention. One approach is to express a candidate gene in the appropriate temporal or spatial manner and examine the phenotypic consequences.

There are currently several available systems intended to regulate the expression of a transgene. Most of these systems have been designed for use in cells in culture and rely on foreign and/or engineered transcription factors that are potentially immunogenic and therefore not ideal for use in animals. These regulatable systems include the Tet-On and Tet-Off (Gossen et al. 1992, Gossen et al. 1995, Baron et al. 2000, Rizzuto et al. 1999, Rendahl et al. 2002) systems, the Mifepristone system (Nordstrom 2003), and Rapamycin system (Rivera et al. 1996, Rivera et al. 1999, Ye et al. 1999, Rivera et al. 2005).

In gene therapy settings, the ability to regulate transgene expression would allow for production of the therapeutic gene product only at the times necessary. For example, in patients with a decreased capacity for erythropoiesis, it would be desirable to express the erythropoietin (EPO) gene at only the desired intervals, and then to silence expression permanently once the underlying cause of anemia has been addressed. This regulation would avoid potential problems associated with gene therapy approaches involving unregulated EPO expression, which could lead to excessive erythropoiesis and polycythemia, and approaches utilizing protein-based transregulators, which are potentially immunogenic. A system for transgene regulation that does not rely on immunogenic transactivators is required.

Recently, much interest has focused on a recently discovered population of non-coding small RNA molecules, termed small interfering RNA (siRNA) and micro RNA (miRNA), and their effect on intracellular processes, particularly gene expression. Micro RNAs (miRNAs) and small interfering RNAs (siRNAs) are small RNAs, about 15-50 nucleotides in length, which play a role in regulating gene expression in eukaryotic organisms through a naturally occurring process termed RNA interference. RNA interference (RNAi) describes a phenomenon whereby the presence of double-stranded RNA (dsRNA) of sequence that is identical or highly similar to a sequence in a target gene messenger RNA (mRNA) results in inhibition of expression of the target gene.

Endogenous miRNAs are transcribed as long primary transcripts (pri-miRNA) or embedded in independent non-coding RNAs or in introns of protein-coding genes. Pri-miRNAs are processed into single-stranded mature miRNAs which guide effector complexes, miRNPs, to their target by base-pairing with target mRNAs. Functional siRNAs and microRNA can be synthesized chemically, transcribed from engineered transgenes or produced naturally

MiRNAs are expressed in a wide variety of organisms ranging including worms (nematodes), insects, plants and animals, including humans. The estimates of the number of miRNA genes vary from 800 to over 2000 with many being conserved across mammalian species. Most animal miRNAs bind to multiple, partially complementary binding sites in the 3′-UTRs of the target genes. However, binding site sequences inserted into either coding or 5′-UTR sequences have also been shown to be functional. The fate of the target mRNA may be decided by the extent of base-pairing to the miRNA. Evidence suggests that miRNA will direct destruction of the target mRNA, gene silencing, if it has perfect or near-perfect complementarity to the target. On the other hand, the presence of multiple, partially complementary sites in the target mRNA may result in translation repression without strongly affecting mRNA levels through inhibit of protein accumulation on the transcript. However, these mRNAs are eventually degraded in the P-bodies.

MiRNAs appear to be a major feature of the gene regulatory networks of animals. Roles for miRNAs have been suggested in development, metabolism, embryogenesis and patterning, differentiation and organogenesis, growth control and programmed cell death, and even human disease, including cancer and inhibition of viral replication. In animals, miRNA has been proposed to primarily fine-tune gene expression and even to dramatically regulate expression of some transcripts. Several miRNAs are expressed in a tissue-specific and developmental stage-specific manner. In addition, it has been shown that the miRNA profiles are altered in a number of cancers. By taking advantage of these characteristics we can use endogenous miRNAs to regulate transgene expression without relying on foreign immunogenic transactivators.

Some of the key properties of miRNAs that make them attractive for use in regulating transgene expression include their ability to strongly suppress the expression of messenger RNAs (mRNAs) containing exact match miRNA binding sites, their tissue and spatial-specific expression patterns, and the availability of antisense miRNA inhibitors. Most importantly, miRNAs are endogenous and non-immunogenic. Their use in regulatory strategies would circumvent possible complications associated with the introduction of protein-based regulators used in most current systems.

### SUMMARY OF THE INVENTION

In a preferred embodiment, we describe compositions and processes for regulated or conditional expression of genes of interest in eukaryotic cells. Insertion of a miRNA binding site into the transcribed region of a gene renders expression of the encoded protein sensitive to miRNA expressed or not expressed in target or non-target cells. The presence in a cell of a miRNA corresponding to the miRNA binding site results in suppression of protein production from the transcript.

In a preferred embodiment, we describe regulatable or conditional expression cassettes comprising: a promoter operatively linked to a gene of interest and one or more miRNA binding sites that are present on the messenger RNA transcribed from the cassette. Preferably, the gene of interest encodes a protein capable of affecting the biological properties of the cell, and can include both therapeutic genes and genes of interest in biological research. A preferred location for the miRNA binding site is the 3′ UTR, however, other sites are not excluded. The cell can be any cell in which miRNA are present and active, including, but not limited to, nematodes, insects, plants and mammals. Additionally, the cell can be in vivo, ex vivo, in situ, or in vitro. In vivo, a preferred target tissue has the potential for secretion of a therapeutic protein.

In a preferred embodiment, we describe an miRNA-regulated expression system comprising: an expression cassette encoding a transgene whose regulation or tissue specific expression is desired and a second expression cassette encoding a repressor of the transgene or inhibitor of the expressed transgene encoded protein wherein the second expression cassette contains a miRNA binding site which regulates expression of the repressor/inhibitor.

In a preferred embodiment, expression from the described expression cassettes can be further regulated by delivering to the cell a miRNA inhibitor. A miRNA inhibitor relieves suppression by interfering with the function of miRNA, preferably in a miRNA-specific manner. A preferred miRNA inhibitor is an antisense oligonucleotide. A preferred antisense oligonucleotide is a locked nucleic acid or an antagomir.

In another preferred embodiment, the described expression cassettes can be used to suppress transcription of a gene in non-target cells by selecting a miRNA binding site which corresponds to miRNA expressed in the non-target cell. In this way, for example, a toxic protein can be delivered to cancer cells without expressing the gene in non-cancer cells.

Any known gene delivery method, including hydrodynamic injection, direct injection, viral infection, gene gun, transfection reagent etc. can be used to deliver the expression cassette to a cell. The expression cassette can be delivered as linear DNA, circular DNA or as part of a linear or circular DNA, such as a plasmid.

In a preferred embodiment, effective miRNA binding sites—miRNA binding site sequence or location within the transcribed miRNA—can be identified by inserting the miRNA binding sites into a reporter gene and delivering the gene to the target tissue. Suppression of the reporter gene indicates presence in the cell of the cognate miRNA and ability of the miRNA to suppress expression of the gene.

Further objects, features, and advantages of the invention will be apparent from the following detailed description when taken in conjunction with the accompanying drawings.

### BRIEF DESCRIPTION OF THE FIGURES

FIG. 1. Illustration of a plasmid containing a sample miRNA-regulated expression cassette. While the mRNA binding site is shown in the 3′ UTR in this example, its location is not limited to the 3′ UTR.

FIG. 2. Graph illustrating tissue-specific miRNA dependent regulation of gene expression from expression cassettes encoding the luciferase gene of interest and the indicated miRNA binding sites. (n=3, error bars represent SD).

FIG. 3. A. Graph illustrating effect of miRNA inhibitor administration on transgene expression from miRNA regulated expression cassettes in mouse liver. Animals received 10 μg of plasmid containing the indicated miRNA binding site together with 10 μg of control 2′-OMe oligonucleotide or an anti-miRNA oligonucleotide specific for the indicated miRNA. Data are plotted as the amount of target Renilla luciferase activity (Rr-Luc) divided by the amount of control firefly luciferase activity (Pp-Luc+) in order to account for differences in delivery efficiency between animals, then scaled to the ratio in animals receiving the no miRNA binding site control plasmid (none). B. Graph illustrating data for the miR-122a regulated plasmid plotted using a smaller scale in order to visualize differences between the control and experimental groups, n=3, error bars represent SD.

FIG. 4. Graph illustrating the specificity of miRNA inhibitor on miRNA-regulated transgene expression in mouse muscle. Plasmids containing expression cassettes with the indicated miRNA binding sites (none, liver specific miR-122a, mutant miR-143 or muscle specific miR-143) were delivered to mouse limb skeletal muscle cells. Two groups received either 2′OMe anti-miR-143 oligonucleotides (anti-miR143, 50 μg) or a control oligonucleotide (control antisense). n=3, error bars represent SEM.

FIG. 5. Graph illustrating alleviation of transgene suppression by co-delivery of antagomirs in liver. Expression cassettes containing the liver specific miR-122a miRNA binding site were delivered alone (−) or with the indicated antisense oligonucleotide. Data are plotted as target Renilla luciferase activity (Rr-Luc) divided by the amount of control firefly luciferase activity (Pp-Luc+) in order to account for differences in delivery efficiency between animals, then scaled to animals receiving control plasmid (none). PS, phosphorothioate linkage; MM, antagomir containing three mismatches. n=3, error bars represent SD.

FIG. 6. Graph illustrating miRNA-regulated EPO expression from mouse liver. 50 ng of the indicated plasmid together with 5 μg of carrier DNA was delivered with or without 25 μg antagomir. Serum EPO was measured by ELISA and plotted on a log scale. N=3, error bars represent SD.

FIG. 6. Graph illustrating hematocrit levels in mice receiving miRNA-regulated EPO expression plasmids. Mice received the indicated plasmids on Day 0. Hematocrit measurements were made in triplicate.

FIG. 7. Graph illustrating hematocrit levels in mice receiving miRNA-regulated EPO expression plasmids. Mice received the indicated plasmids on Day 0. Hematocrit measurements were made in triplicate.

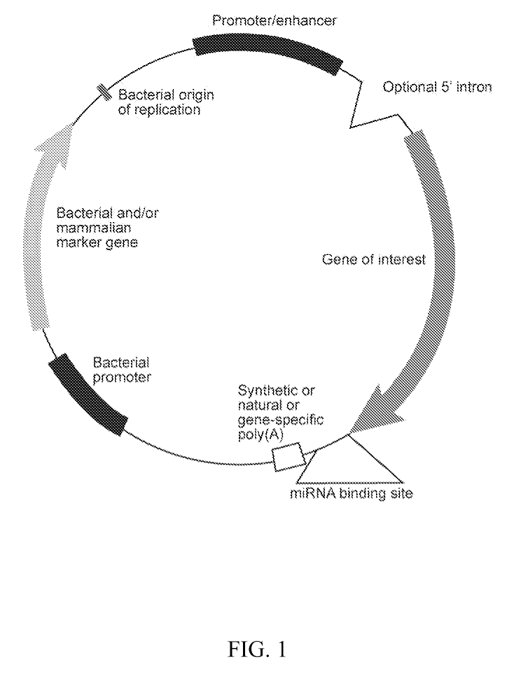

### DETAILED DESCRIPTION OF THE INVENTION

The disclosed invention provides an expression system for conditional or regulated expression of an encoded transgene based the presence or absence of a miRNA in a cell or tissue. The expression of the transgene can be further regulated by administration, either simultaneously or sequentially, of miRNA-specific miRNA inhibiting molecules.

In one embodiment, an expression cassette is described comprising: a gene of interest or a cloning site into which a gene of interest can be inserted, a promoter/enhancer which directs expression of the gene (long term expression), and one or more mRNA binding sites. The described expression system can be used to facilitate regulated or conditional expression in cells in vivo, in vitro, or ex vivo. The regulated or conditional expression may be used to achieve tissue specific expression or developmentally regulated expression of a gene inserted into the expression cassette.

A miRNA binding site is a nucleotide sequence which is complementary or partially complementary to at least a portion of a miRNA. The sequence can be a perfect match, meaning that the binding site sequence has perfect complementarity to its cognate miRNA (perfect miRNA binding site). Alternatively, the sequence can be partially complementary to an expressed miRNA, meaning that one or more mismatches may occur when the cognate miRNA is base paired to the binding site (imperfect mRNA binding site). Partially complementary binding sites preferably contain perfect or near perfect complementarity to the seed region of the miRNA. The seed region of the miRNA consists of the 5′ region of the miRNA from about nucleotide 2 to about nucleotide 8 of the miRNA. For naturally occurring miRNAs and target genes, mRNAs with perfect complementarity to a mRNA sequence direct degradation of the mRNA through the RNA interference pathway while mRNAs with imperfect complementarity to the target mRNA direct translational control (inhibition) of the mRNA. The invention is not limited by which pathway is ultimately utilized by the miRNA in inhibiting expression of the transgene or encoded protein.

In one embodiment the described expression cassettes contain miRNA binding sites with perfect complementarity to their cognate miRNAs (perfect mRNA binding sites). Perfect complementarity of a miRNA with its target mRNA sequence has been shown to act like small interfering RNAs (siRNAs) and cause target mRNA cleavage (Hutvagner et al. 2002, Zeng et al. 2003). The presence of a single, perfectly matched miRNA binding site in the transcribed mRNA is sufficient to dramatically inhibit expression of the gene. Thus, transgenes can be suppressed by endogenous miRNA by placing a single exact match miRNA binding site within the transcribed mRNA sequence of the transgene. However, the invention is not limited to expression cassettes containing a simple perfectly matched miRNA binding site. In another embodiment, the described expression cassettes contain one or more miRNA binding sites with imperfect complementarity (imperfect miRNA binding sites). In yet another embodiment, the expression cassettes may contain both perfect and imperfect miRNA binding sites. Expression cassettes can therefore be tailored to result in varying levels of regulation by using single perfect, multiple perfect, single imperfect, multiple imperfect or a combination of perfect and imperfect miRNA binding sites. Further, miRNA binding sites for different cognate miRNAs may also be used, therefore permitting a gene to be regulated by multiple miRNAs. A preferred location for the miRNA binding site is the 3′UTR. However, binding site sequences inserted into either coding or 5′-UTR sequences may also be used.

The choice of miRNA binding site is determined by the desired expression pattern. The presence of an endogenous miRNA in a cell will inhibit expression of a gene which contains a cognate miRNA binding site(s). For expression of the gene of interest to be inhibited in a given cell-type, a miRNA binding site that is recognized by a miRNA present in that cell-type is chosen.

The gene of interest can be any gene which encodes a protein of interest and includes both therapeutic genes and genes of biological interest. The gene of interest is meant to include a gene whose expression in a cell effects the biological properties of the cell, tissue or organism. The gene of interest is meant to exclude genes generally recognized in the art as reporter genes. Excluded reporter genes include luciferases, fluorescent proteins such as green fluorescent protein, β-galactosidase, chloramphenicol acetyl transferase, secreted alkaline phosphatase, and the like. However, reporter genes can be used to test the efficacy of a miRNA binding site or of a given expression cassette. The cassette is tested by substituting the reporter gene for the gene of interest.

A promoter directs transcription of a gene. Promoters are generally located upstream of a transcribed gene and provide binding sites for components of RNA polymerase or factors which affect the binding or activity of RNA polymerase. A promoter often contains a TATA box sequence and/or an initiator sequence. An enhancer is a DNA sequence to which transcription factors/activators bind to increase expression of a gene. The sequence may be located upstream, downstream, within an intron, in 5′ or 3′ untranslated regions, or within the coding sequence of a gene. The transcription activators affect recruitment of components of the RNA polymerase complex to the promoter, can affect recruitment of chromatin remodeling factors and RNA processing or export factors, or affect processivity of the RNA polymerase. For the purposes of the present invention, the term promoter includes both promoters and enhancers. Promoters can be strong or weak and constitutive or regulated. Regulated promoters can provide tissue-specific gene expression, developmentally regulated gene expression, or conditionally regulated gene expression. A preferred promoter is one capable of long term sustained expression in the target cell type.

In another embodiment the expression system comprises an expression cassette encoding a transgene whose regulation or tissue specific expression is desired and a second expression cassette encoding a repressor of the transgene or inhibitor of the expressed transgene encoded protein. The second expression cassette further contains a miRNA binding site which regulates expression of the repressor/inhibitor. By placing a miRNA binding site in the transcribed mRNA of the repressor/inhibitor gene, expression of the repressor/inhibitor is made dependent on the presence or absence of the cognate miRNA in the cell. If the plasmid is delivered to a cell of interest and the miRNA is present in the cell, the miRNA binds and causes inhibition of expression of the repressor/inhibitor mRNA. By reducing or eliminating expression of the repressor/inhibitor, expression or activity of the transgene is increased. Expression of the transgene in non-target cells is reduced because of the absence of the miRNA, resulting in expression of the repressor/inhibitor and therefore repression or inhibition of the transgene.

The described expression systems can be used in combination with miRNA inhibitors. Inhibition of the miRNA relieves inhibition of the transgene. Known mRNA inhibitors include antisense molecules such as antagomirs. MiRNA inhibitors can reduce or prevent production of a specific miRNA or inhibit binding of a miRNA to a miRNA-binding site. Stability or persistence of the miRNA inhibitor will determine the length the time the inhibitor is effective. Loss of the inhibitor results in inhibition of the transgene.

RNA oligonucleotides that are perfectly complementary to the target miRNA, antisense miRNA inhibitors, have been shown to inhibit miRNA function through stoichiometric binding to the miRNA (Hutvagner et al. 2004, Meister et al. 2004, Cheng et al. 2005). Antisense miRNA inhibitors have also been shown to be effective in vivo (Krutzfeldt et al. 2005, Esau et al. 2006). Antisense miRNA oligonucleotide containing 2′-OMe substitutions throughout, phosphorothioate linkages in the first two 5′ and last three 3′ nucleotides, and a cholesterol moiety attached at the 3′ end have been termed antagomirs. The inhibitory effect the antagomir can last longer than 20 days and is effective in multiple tissue types.

The describe expression system can be used for targeting expression to specific cells or tissues for expression of beneficial genes. For example, a gene delivery procedure could deliver the gene of interact to multiple cells, including target and non-target cells. The presence of a miRNA binding site for a miRNA absent from target cell but present in non-target cells would result in expression in target cells and repression in non-target cells. As an example, for an expression cassette encoding vascular endothelial growth factor (VEGF), the presence of a miRNA binding site could be used to limit the population of target cells, therefore limiting the overall level of expression of this secreted protein.

The described expression system can also be used to target toxic proteins to certain cells, such as cancers cells, to eliminate those cells. The absence of a cognate miRNA in the target cell, and presence of the miRNA in non-target cells would limit expression to the target cells. Tumor necrosis factor-α (TNFα) is an example of a toxic protein.

The pattern of expression can be effectively reversed if a regulator/inhibitor of the gene of interest is placed under transcriptional regulation of a miRNA. As an example illustrating the process, an expression cassette can be constructed that encodes TNFα and a TNFα repressor such as heat shock factor 1 (HSF-1). A miRNA binding site is placed in the HSF-1 gene transcript such that binding of a miRNA represses its expression. Thus, presence of the cognate miRNA in the target cell inhibits expression of the inhibitor, leading to expression of TNFα. Conversely, absence of the cognate miRNA in non-target cell results in expression of the inhibitor which in turn inhibits TNFα.

By administering miRNA inhibitors, regulation of the expression cassettes can be further modulated. It may be desirable to limit expression of beneficial genes such a VEGF and erythropoietin (EPO). These genes can be important therapeutically, however, their over-expression has toxic effects. VEGF is used to increase blood flow in patients with peripheral arterial occlusive disease. However, over production of VEGF can lead to the production of hemangiomas. EPO increases red blood cell production and is used to treat anemia. However, over production of EPO causes a deleterious thickening of the blood. By making their expression sensitive to endogenous miRNAs, the use of miRNA inhibitors allows the production of these genes to be modulated after delivery. Administration of an inhibitor leads to increase production of the protein while absence of an inhibitor leads to decrease protein production. The miRNA inhibitor thus serves as an inducer to expression.

MicroRNAs have been identified using microarray and Northern blot analyses. Using these methods, 71 miRNAs have been shown to have detectable expression in skeletal muscle of mice. In addition, microRNA sensor plasmids have been used to detect expression of functional miRNAs in cells in culture and in transiently transgenic mouse embryos (Smirnova et al. 2005, Mansfield et al. 2004). In muscle cells, miR-1 enhances myogenesis and myofiber formation and miR-133 promotes myoblast proliferation. In pancreatic islet cells, miR-375 is involved in glucose stimulated insulin secretion. In liver cells, miR-122a is involved in cholesterol homeostasis. Lists of known miRNA sequences can be found in databases maintained by research organizations such as the Wellcome Trust Sanger Institute. The current number of known or suspected mouse miRNAs is more that 200 (miRBase release 7.1).

The term expression cassette refers to a naturally, recombinantly, or synthetically produced nucleic acid molecule that is capable of expressing a gene or genetic sequence in a cell. An expression cassette typically includes a promoter and a sequence encoding one or more proteins or subunit(s) of a protein. Optionally, the expression cassette may include transcriptional enhancers, non-coding sequences, splicing signals and introns, internal ribosome entry sites (IRES), transcription termination signals, and polyadenylation signals. As described above, the expression cassette may also include a miRNA binding site.

The term gene generally refers to a nucleic acid sequence that comprises coding sequences necessary for the production of a nucleic acid (e.g., miRNA or antisense nucleic acid) or a polypeptide (protein) or protein precursor. A polypeptide can be encoded by a full length coding sequence or by any portion of the coding sequence so long as the desired activity or functional properties (e.g., enzymatic activity, ligand binding, signal transduction) of the full-length polypeptide or fragment are retained. In addition to the coding sequence, the term gene may also include, in proper contexts, the sequences located adjacent to the coding region on both the 5′ and 3′ ends which correspond to the full-length mRNA (the transcribed sequence) or all the sequences that make up the coding sequence, transcribed sequence and regulatory sequences. The sequences that are located 5′ of the coding region and which are present on the mRNA are referred to as 5′ untranslated region (5′ UTR). The sequences that are located 3′ or downstream of the coding region and which are present on the mRNA are referred to as 3′ untranslated region (3′ UTR). The term gene encompasses synthetic, recombinant, cDNA and genomic forms of a gene. A genomic form or clone of a gene contains the coding region interrupted with non-coding sequences termed introns, intervening regions or intervening sequences. Introns are segments of a gene which are transcribed into nuclear RNA. Introns may contain regulatory elements such as enhancers. Introns are removed or spliced from the nuclear or primary transcript; introns therefore are absent in the mature mRNA transcript. Regulatory sequences include, but are not limited to, promoters, enhancers, transcription factor binding sites, polyadenylation signals, internal ribosome entry sites, silencers, insulating sequences, matrix attachment regions. Non-coding sequences may influence the level or rate of transcription and/or translation of the gene. Covalent modification of a gene may influence the rate of transcription (e.g., methylation of genomic DNA), the stability of mRNA (e.g., length of the 3′ polyadenosine tail), rate of translation (e.g., 5′ cap), nucleic acid repair, nuclear transport, and immunogenicity. Gene expression can be regulated at many stages in the process. Up-regulation or activation refers to regulation that increases the production of gene expression products (i.e., RNA or protein), while down-regulation or repression refers to regulation that decreases production. Molecules (e.g., transcription factors) that are involved in up-regulation or down-regulation are often called activators and repressors, respectively.

Long term expression means that the gene is expressed for greater than 2 weeks, greater than 4 weeks, greater than 8 weeks, greater than 20 weeks, greater than 30 weeks, or greater than 50 weeks with less than a 10-fold decrease in expression from day 1. Expression in liver cells in vivo from typical CMV promoter driven gene expression cassettes typically drops by up to 1000-fold after 7 days. Expression for longer than a few weeks may require not eliciting an immune response to the expressed gene product, which is independent of the promoter/enhancer elements of the expression cassette. An immune response can be avoided or minimized by using immunosuppressive drugs, immune compromised animals, or expressing a gene product that is minimally or non-immunogenic. In one embodiment, the miRNA sensor plasmid that contains elements that allow for long-term expression of a transgene in liver as described in U.S. application Ser. No. 10/229,786 (U.S. application Ser. No. 10/229,786 is incorporated herein by reference)

The term polynucleotide, or nucleic acid, is a term of art that refers to a polymer containing at least two nucleotides. Nucleotides are the monomeric units of polynucleotide polymers. Polynucleotides with less than 120 monomeric units are often called oligonucleotides. Natural nucleic acids have a deoxyribose- or ribose-phosphate backbone. An artificial or synthetic polynucleotide is any polynucleotide that is polymerized in vitro or in a cell free system and contains the same or similar bases but may contain a backbone of a type other than the natural ribose-phosphate backbone. These backbones include: PNAs (peptide nucleic acids), phosphorothioates, phosphorodiamidates, morpholinos, and other variants of the phosphate backbone of native nucleic acids. Bases include purines and pyrimidines, which further include the natural compounds adenine, thymine, guanine, cytosine, uracil, inosine, and natural analogs. Synthetic derivatives of purines and pyrimidines include, but are not limited to, modifications which place new reactive groups such as, but not limited to, amines, alcohols, thiols, carboxylates, and alkylhalides. The term base encompasses any of the known base analogs of DNA and RNA. The term polynucleotide includes deoxyribonucleic acid (DNA) and ribonucleic acid (RNA) and combinations on DNA, RNA and other natural and synthetic nucleotides.

The polynucleotide may contain sequences that do not serve a specific function in the target cell but are used in the generation of the polynucleotide. Such sequences include, but are not limited to, sequences required for replication or selection of the polynucleotide in a host organism. A polynucleotide may also include sequences which allow replication of the polynucleotide in mammalian cells.

Small RNAi molecules include RNA molecules less that about 50 nucleotides in length and include siRNA and miRNA. SiRNA comprises a double stranded structure typically containing 15-50 base pairs and preferably 19-27 base pairs and having a nucleotide sequence identical or nearly identical to an expressed target gene or RNA within the cell. An siRNA may be composed of two annealed polynucleotides or a single polynucleotide that forms a hairpin structure. MicroRNAs (miRNAs) are small non-coding polynucleotides that direct destruction or translational repression of their mRNA targets.

Antisense polynucleotides comprise sequence that is complimentary to a gene or RNA and can base pair to a gene, RNA or portion thereof. Antisense polynucleotides include, but are not limited to: morpholinos, 2′-O-methyl polynucleotides, DNA, RNA and the like.

A therapeutic effect of the protein in attenuating or preventing the disease state can be accomplished by the protein either staying within the cell, remaining attached to the cell in the membrane or being secreted and dissociating from the cell where it can enter the general circulation and blood. Secreted proteins that can be therapeutic include hormones, cytokines, growth factors, clotting factors, anti-protease proteins (e.g. alpha-antitrypsin) and other proteins that are present in the blood. Proteins on the membrane can have a therapeutic effect by providing a receptor for the cell to take up a protein or lipoprotein. For example, the low density lipoprotein (LDL) receptor could be expressed in hepatocytes and lower blood cholesterol levels and thereby prevent atherosclerotic lesions that can cause strokes or myocardial infarction. Therapeutic proteins that stay within the cell can be enzymes that clear a circulating toxic metabolite as in phenylketonuria. They can also cause a cancer cell to be less proliferative or cancerous (e.g. less metastatic). A protein within a cell could also interfere with the replication of a virus.

We have disclosed gene expression achieved from reporter genes in specific tissues. The terms therapeutic and therapeutic results are defined in this application as a nucleic acid which is transfected into a cell, in vivo, resulting in a gene product (e.g. protein) being expressed in the cell or secreted from the cell. Levels of a gene product, including reporter (marker) gene products, are measured which then indicate a reasonable expectation of similar amounts of gene expression by transfecting other nucleic acids. Levels of treatment considered beneficial by a person having ordinary skill in the art of gene therapy differ from disease to disease, for example: Hemophilia A and B are caused by deficiencies of the X-linked clotting factors VIII and IX, respectively. Their clinical course is greatly influenced by the percentage of normal serum levels of factor VIII or IX: <2%, severe; 2-5%, moderate; and 5-30% mild. This indicates that in severe patients an increase from 1% to 2% of the normal level can be considered beneficial. Levels greater than 6% prevent spontaneous bleeds but not those secondary to surgery or injury. A person having ordinary skill in the art of gene therapy would reasonably anticipate beneficial levels of expression of a gene specific for a disease based upon sufficient levels of marker gene results. In the hemophilia example, if marker genes were expressed to yield a protein at a level comparable in volume to 2% of the normal level of factor VIII, it can be reasonably expected that the gene coding for factor VIII would also be expressed at similar levels.

### EXAMPLES

### Example 1

Tissue-specific miRNA-mediated expression cassette. In order to demonstrate that miRNAs can be used to suppress transgene expression in cells in vivo, a plasmid was made that contained an expression cassette encoding a reporter gene and a various miRNA binding sites. The test the expression system, a modified PSICHECK™-2 vector (Promega, Madison, Wis.) was used. This commercially available plasmid encodes both the Renilla and firefly luciferase genes and was originally developed for use in determining the activity of candidate siRNAs. For in vivo studies, the Renilla luciferase gene acts as the gene of interest while the firefly luciferase gene served as an internal control that permitted normalization of delivery efficiency.

Various perfect miRNA binding sites were inserted into XhoI/NotI sites in the 3′ UTR of the Renilla luciferase gene. The miRNA binding sites were selected to bind with miRNAs known to be expressed in muscle and liver target tissues. Sequences of mature mouse miRNAs were acquired from the miRBase Sequence Database (http://microrna.sanger.ac.uk/sequences/). Five known miRNA sequences were chosen and their exact DNA complements and respective antisense sequences were obtained from IDT (Coralville, Iowa), Table 1. All oligonucleotides were ordered with 5′ Xho I linkers and 3′ Not I linkers to allow ligation into the PSICHECK™-2 in the proper orientation. Equal molar amounts of each oligonucleotide pair were annealed and ligated into the vector.

TABLE 1

| | |
|---|---|
| MRT-122S | TCGAGACAAACACCATTGTCACACTCCAGC |
| MRT-122AS | GGCCGCTGGAGTGTGACAATGGTGTTTGTC |
| MRT-192S | TCGAGGGCTGTCAATTCATAGGTCAGGC |
| MRT-192AS | GGCCGCCTGACCTATGAATTGACAGCCC |
| MRT-1S | TCGAGTACATACTTCTTTACATTCCAGC |
| MRT-1AS | GGCCGCTGGAATGTAAAGAAGTATGTAC |
| MRT-18S | TCGAGTATCTGCACTAGATGCACCTTAGC |
| MRT-18AS | GGCCGCTAAGGTGCATCTAGTGCAGATAC |
| MRT-143S | TCGAGTGAGCTACAGTGCTTCATCTCAGC |
| MRT-143AS | GGCCGCTGAGATGAAGCACTGTAGCTCAC |

MRT = miRNA binding site oligonucleotide; numbers refer to the miRNA as listed in the Sanger Institute miRNA Registry.
S = sense strand containing sequence complementary to that of corresponding endogenous miRNA according to standard convention.
AS = antisense strand which contains sequence complementary to the corresponding sense strand.

### Example 2

Tissue specific miRNA-mediated gene suppression of a transgene in muscle and liver in vivo. Expression cassettes were delivered to mouse liver and muscle cells in vivo via hydrodynamic injection (U.S. Pat. No. 6,627,616 and US-2004-0242528). Five miRNA regulated Renilla luciferase expression cassette constructs were delivered separately to liver or limb skeletal muscle cells and monitored for transgene expression. Included were expression cassettes containing the liver specific miRNA-122a mRNA binding site and the muscle-specific miR-1 miRNA binding site. For delivery to liver, 10 μg of plasmid was injected. Livers were harvested one day after injection. For delivery to skeletal muscle, 20 μg of plasmid was injected and muscle from the injected limb was harvested two days after injection.

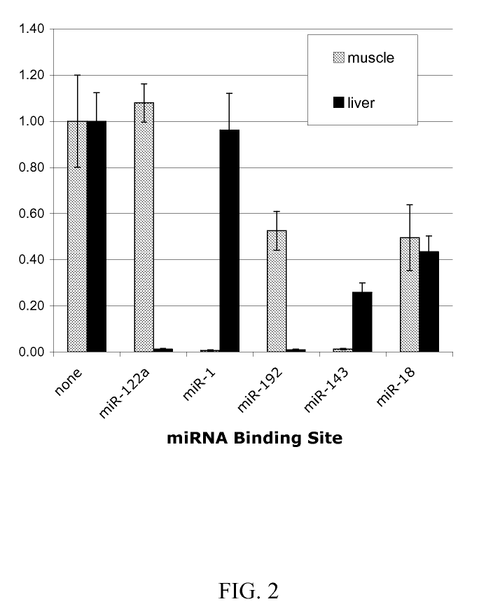

After harvest and homogenization, tissue extracts were assayed for both the Renilla luciferase and firefly luciferase activity. Activity of Renilla luciferase was divided by the activity of firefly luciferase in order to compensate for differences in delivery efficiency between animals. Data was normalized to animals receiving an expression cassette without a mRNA binding site. According to published data, the miRNAs, miR-122a and miR-192, are highly expressed in liver, but not detected in skeletal muscle. Conversely, mRNA miR-1 is highly expressed in skeletal muscle, but not detected in liver. As expected, and shown in FIG. 2, Renilla luciferase expression was nearly completely inhibited in liver, but not muscle, in expression cassettes containing the miR-122a and miR-192 miRNA binding sites. In expression cassettes containing the miR-1 miRNA binding site, Renilla luciferase expression was nearly completely inhibited in muscle but unaffected in liver. Expression cassettes containing the miR-143 miRNA binding sites showed greater inhibition in liver than in muscle. This result correlates with microarray and Northern data, which indicate that higher miRNA-143 expression in liver than in muscle. For expression cassettes containing the miR-18 mRNA binding site, a moderate level of inhibition is observed in both liver and muscle. The miRNA miR-18 has not been previously detected in these tissues. From these results, it is predicted that miR-18 is expressed at low levels in liver and muscle cells in mouse.

### Example 3

Inhibition of miRNA-mediated transgene suppression. MiRNA inhibitors can be used to inhibit miRNA activity and to relieve suppression of transgene expression at desired times.

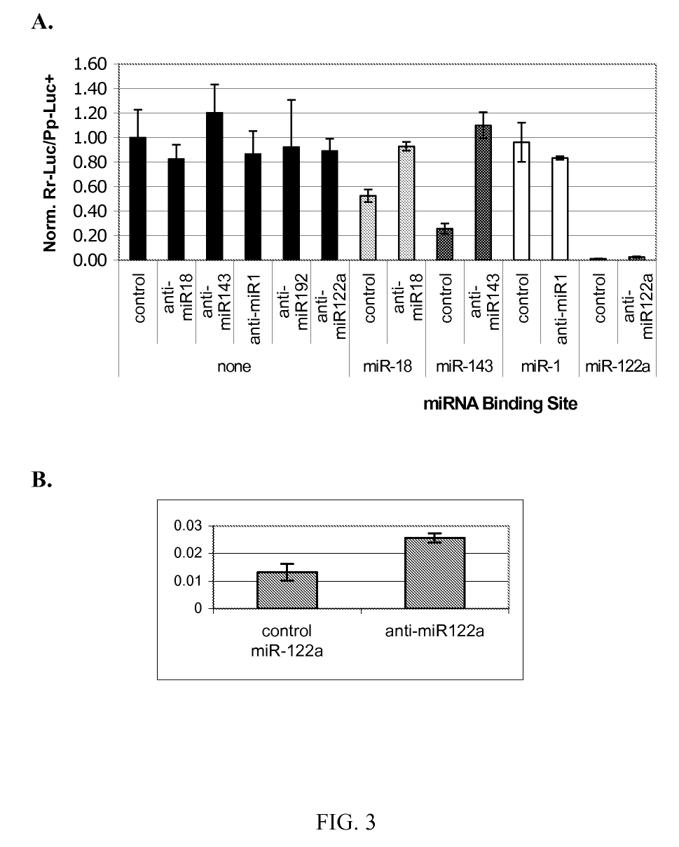

A. Inhibition of miRNA function in liver using 2′-OMe substituted antisense oligonucleotides. Studies have shown that miRNAs can be inhibited by oligonucleotides containing 2′-O-methyl (2′-OMe) substitutions having the antisense sequence to the mature miRNA (Alvarez-Garcia et al. 2005, Chen et al. 2006). Inhibition was shown to be due to stoichiometric binding to the miRNA. In order to test the ability of antisense to relieve the miRNA suppression of transgene expression in vivo, 10 μg of plasmid containing expression cassettes with the binding sites for either miRNA-18, 143, or 122a were co-delivered to liver by hydrodynamic tail vein injection with 10 μg of the indicated 2′-O-methyl (2′-OMe) antisense oligonucleotides or a non-specific antisense control. Controls also included delivery of miRNA-1 regulated plasmid, which is not inhibited in liver due to the lack of miR-1 in this organ, and plasmid containing no miRNA binding site. Livers were harvested one day after injection and extracts assayed for Renilla and firefly luciferase activities. The results are shown in FIG. 3.

Antisense oligonucleotides to the miRNAs were able to provide total relief of inhibition when co-delivered with miR-18 and miR-143 containing expression cassettes. In the case of miR-122a, inhibition was evident but incomplete (see inset graph in FIG. 3). Incomplete inhibition by antisense could be due to high levels of miRNA in liver. It has been reported that miRNA-122a is highly expressed in hepatocytes, with more than 50,000 copies per cell (Krutzfeldt et al. 2005). It is possible that antisense molecules containing other types of substitutions or modifications would be superior inhibitors of miRNA function. Three mutations in the binding site of the miR-122a regulated plasmid abolished inhibition (data not shown), implying that inhibition is miR-122a specific.

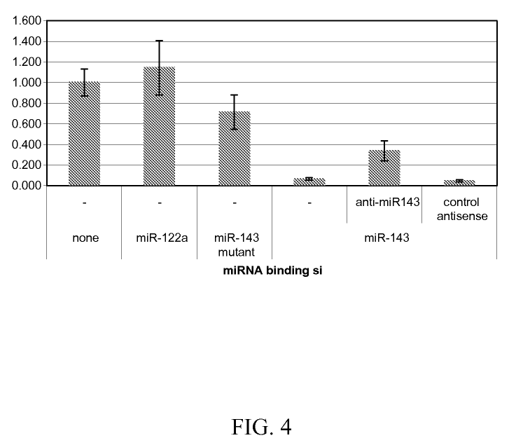

B. Inhibition of miRNA function in skeletal muscle using 2′-OMe substituted antisense oligonucleotides. We examined whether miRNA function could be inhibited in muscle by co-delivery of antisense oligonucleotides. Plasmids containing miR-143 miRNA regulated expression cassettes were delivered to limb skeletal muscle with or without 2′-OMe antisense inhibitor using hydrodynamic limb vein injection. Control plasmids without a miRNA binding site, with a liver-specific miR-122a miRNA biding site or containing a miR-143 binding site mutated at base positions 3, 7, and 10 were also delivered. Results are shown in FIG. 4. As also shown in FIG. 2, strong suppression of reporter gene expression in muscle was observed in plasmid harboring the miR-143 site relative to those containing no miRNA binding site or the binding site for miR-122a. The suppression was specific to miR-143 as the presence of three mutations in the binding site abolished suppression. Suppression was inhibited by co-delivery of antisense miR-143 2′-OMe oligonucleotide. Delivery of more miRNA inhibitor or more effective miRNA inhibitor would be expected to result in greater alleviation of miR-143 dependent suppression.

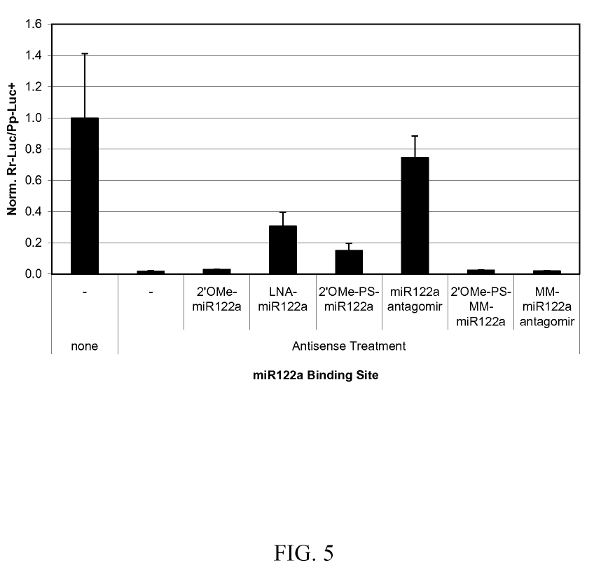

C. Comparison of antisense chemistries for miRNA inhibition in vivo. Because inhibition of miR-143 in muscle and miR-122a in liver appeared to be incomplete, the in vivo effectiveness of antisense miRNA inhibitors containing locked nucleic acid (LNA) antisense modifications or antagomirs were tested. LNAs contain a bridge between the 2′-O and the 4′-position via a methylene linker that “locks” it into a C3′-endo (RNA) sugar conformation. LNAs have been used previously to inhibit miRNA function. The results for liver are shown in FIG. 5. The LNA modification was 10-fold more effective than the 2′-OMe substituted oligonucleotide, resulting in recovery of miRNA regulated reporter gene activity to 30% of control levels. Co-delivery of the antagomir resulted in even greater recovery of reporter gene expression, with levels reaching those of the reporter gene without the miRNA binding site. Using the antagomir, greater than 40-fold dynamic range of expression was observed. The fact that full activity can be recovered using the miR-122a antagomir is evidence that suppression is in fact due to miR-122a, and not due to other factors. The degree of relief from suppression may depend on the potency of each antagomir and the expression level of the individual miRNA.

### Example 4

Regulation of EPO expression in liver. Although constitutive EPO expression could be desirable for some patients, such as those with end-stage renal failure or AIDS-related anemia, there are risks associated with uncontrolled EPO expression. In addition, some anemia patients would not require life-long EPO gene therapy. Furthermore, it may be most desirable to produce EPO at desired intervals. Thus, a more desirable gene therapy treatment for anemia would incorporate controlled EPO expression. The described regulated expression systems can by utilized in therapeutic gene therapy applications where expression of the transgene requires regulation. MiRNA-regulated gene expression would allow for expression of a delivered EPO gene for a controlled period. We show that EPO expression can be controlled by endogenous miRNAs by delivery of an EPO expression cassette containing a miRNA binding site.

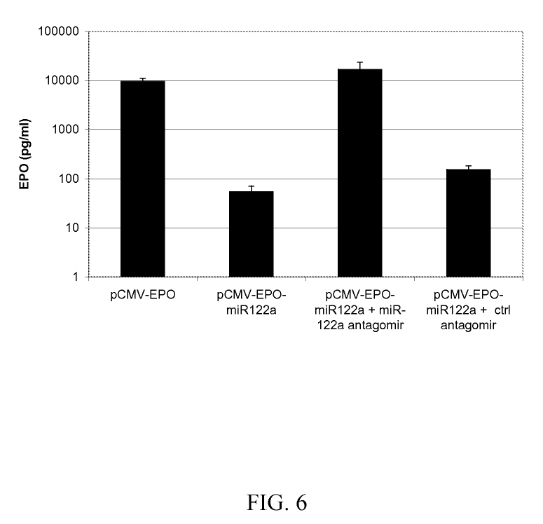

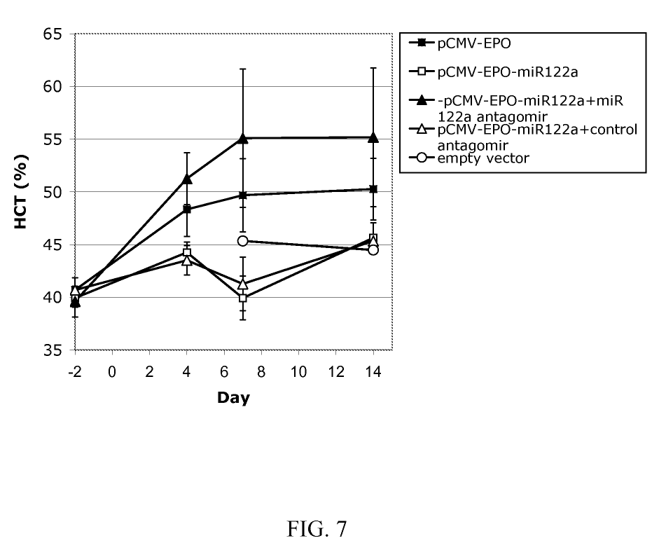

To demonstrate the utility of the described expression cassettes, a miRNA binding site for the liver-specific miR-122a was inserted into the 3′ UTR of the EPO gene. The enhancer used in the construct was the CMV enhancer, which gives very high initial expression but is then inactivated after 18-24 hours in the liver. The resulting expression cassette was delivered to mouse liver cells in vivo using hydrodynamic tail vein injection. Further, the expression cassettes were delivered either with or without miR-122a antagomir. The amount of EPO in the bloodstream and hematocrit were measured at various time points after gene delivery. The results, shown in FIGS. 6 and 7, show that EPO expression one day after delivery was high in control constructs lacking the miR-122a miRNA binding site. We observed an increase in hematocrit over time in animals receiving the control pCMV-EPO construct that did not contain a miRNA binding site. In animals receiving the pCMV-EPO-miR122a construct, no increase was observed in hematocrit levels relative to naive controls. When the miRNA-122a miRNA binding site was present EPO expression was suppressed 180-fold. In contrast, animals receiving the pCMV-EPO-miR122a construct plus the miR-122a antagomir displayed an increase in hematocrit similar to that observed in animals receiving pCMV-EPO. Co-delivery of a control antagomir did not relieve suppression. These results indicate that miRNA and antisense miRNA inhibitors can be used to regulate expression of a therapeutically relevant gene to biological effect. The described system enables the use of endogenous miRNAs to suppress levels of transgene expression to below biologically relevant levels. Suppression can then be relieved by administration of a miRNA inhibitor, enabling transgene expression to biological relevant levels. Repeat dosing of inhibitor would provide for controlled intervals of EPO expression.

The foregoing is considered as illustrative only of the principles of the invention. Furthermore, since numerous modifications and changes will readily occur to those skilled in the art, it is not desired to limit the invention to the exact construction and operation shown and described. Therefore, all suitable modifications and equivalents fall within the scope of the invention.

We claim:

1. An expression cassette for regulated expression of a gene of interest comprising: a promoter operatively linked to the gene of interest and a miRNA binding site wherein the miRNA binding site is present on the messenger RNA (mRNA) transcribed from the expression cassette.

2. The expression cassette of claim 1 wherein the miRNA binding site consists of a perfect miRNA binding site.

3. The expression cassette of claim 1 wherein the miRNA binding site consists of an imperfect miRNA binding site.

4. The expression cassette of claim 1 wherein the miRNA binding site is present in the 3′ UTR of the mRNA.

5. The expression cassette of claim 1 wherein the promoter consists of a promoter capable or long term expression in a target cell.

6. A plasmid for regulated expression of a gene of interest comprising: a first expression cassette encoding the gene of interest and a second expression cassette encoding a regulator or inhibitor of the gene of interest wherein a miRNA binding site is present on the messenger RNA (mRNA) transcribed from the second expression cassette.

7. The plasmid of claim 6 wherein the miRNA binding site consists of a perfect miRNA binding site.

8. The expression cassette of claim 6 wherein the miRNA binding site is present in the 3′ UTR of the mRNA.

9. The expression cassette of claim 6 wherein the first and second expression cassettes contain promoters capable or long term expression in a target cell.

10. A method for regulated expression of a gene of interest comprising: delivering to a cell an expression cassette containing a promoter operatively linked to the gene of interest and a miRNA binding site wherein the miRNA binding site is present on the messenger RNA (mRNA) transcribed from the expression cassette.

11. The method of claim 10 wherein the miRNA binding site consists of a perfect miRNA binding site.

12. The method of claim 10 wherein the cell does not express a miRNA corresponding to the miRNA binding site.

13. The method of claim 10 wherein the cell expresses a miRNA corresponding to the miRNA binding site.

14. The method of claim 13 further comprising delivering to the cell a miRNA inhibitor.

15. The method of claim 14 wherein the miRNA inhibitor comprises an antisense oligonucleotide.

16. The method of claim 15 wherein the antisense oligonucleotide is selected from the groups consisting of: locked nucleic acid and antagomir.

17. The method of claim 10 wherein the gene of interest encodes a toxic protein.

18. The method of claim 10 wherein the gene of interest consists of a regulator or inhibitor of a second gene of interest and further comprising delivering to the cell a second expression cassette encoding the second gene of interest.

19. The method of claim 17 further comprising delivering to the cell a miRNA inhibitor.


SEQUENCE LISTING (10 sequences)

SEQ ID NO: 1 - DNA, 30 bp, Mus musculus
tcgagacaaacaccattgtcacactccagc

SEQ ID NO: 2 - DNA, 30 bp, Mus musculus
ggccgctggagtgtgacaatggtgtttgtc

SEQ ID NO: 3 - DNA, 28 bp, Mus musculus
tcgagggctgtcaattcataggtcaggc

SEQ ID NO: 4 - DNA, 28 bp, Mus musculus
ggccgcctgacctatgaattgacagccc

SEQ ID NO: 5 - DNA, 28 bp, Mus musculus
tcgagtacatacttctttacattccagc

SEQ ID NO: 6 - DNA, 28 bp, Mus musculus
ggccgctggaatgtaaagaagtatgtac

SEQ ID NO: 7 - DNA, 29 bp, Mus musculus
tcgagtatctgcactagatgcaccttagc

SEQ ID NO: 8 - DNA, 29 bp, Mus musculus
ggccgctaaggtgcatctagtgcagatac

SEQ ID NO: 9 - DNA, 29 bp, Mus musculus
tcgagtgagctacagtgcttcatctcagc

SEQ ID NO: 10 - DNA, 29 bp, Mus musculus
ggccgctgagatgaagcactgtagctcac